(19) United States
(12) Patent Application Publication
(10) Pub. No.: US 2026/0085052 A1
(43) Pub. Date: Mar. 26, 2026

(54) OXIDATION OF FURAN DERIVATIVE

(71) Applicant: Furanix Technologies B.V., Amsterdam (NL)

(72) Inventors: David Lee Sikkenga, Wheaton, IL (US); Jan Hendrik Blank, Amsterdam (NL)

(21) Appl. No.: 19/110,145

(22) PCT Filed: Sep. 22, 2023
(86) PCT No.: PCT/EP2023/076176

§ 371 Date: Mar. 10, 2025

(30) Foreign Application Priority Data
Sep. 23, 2022 (EP) 22197516.2

(51) Int. Cl. C07D 307/46

(52) U.S. Cl. CPC C07D 307/46

(57) ABSTRACT

Process for producing 2,5-furandicarboxylic acid includes (i) contacting in an oxidation unit a furan derivative with oxidant and solvent in the presence of a catalyst system to obtain intermediate product including 2,5-furandicarboxylic acid, (ii) contacting in a post-oxidation unit the intermediate product with oxidant to which post-oxidation unit oxidizable compound is added thereby obtaining crude product, and (iii) separating the crude product into 2,5-furandicarboxylic acid and mother liquor containing solvent where the oxidizable compound is selected from tetrahydrofuran containing compounds having from 4 to 10 carbon atoms and alcohols having from 2 to 8 carbon atoms and mixtures thereof.

### CROSS-REFERENCE TO RELATED APPLICATIONS

This application is the National Stage of International Application No. PCT/EP2023/076176, filed Sep. 22, 2023, which claims the benefit of European Application No. 22197516.2, filed Sep. 23, 2022, the contents of which is incorporated by reference herein.

### TECHNICAL FIELD

The present invention relates to a process for producing 2,5-furandicarboxylic acid by contacting a furan derivative with oxidant and a catalyst system followed by post-oxidation.

### BACKGROUND

2,5-Furandicarboxylic acid (FDCA) is known in the art to be a highly promising building block for replacing petroleum-based monomers in the production of high performance polymers. In recent years, 2,5-furandicarboxylic acid and the novel plant-based polyester polyethylenefuranoate (PEF), a completely recyclable plastic with superior performance properties compared to today's widely used petroleum-based plastics, have attracted a lot of attention. These materials could provide a significant contribution to reducing the dependence on petroleum-based polymers and plastics, while at the same time allowing for a more sustainable management of global resources. Comprehensive research was conducted in the field to arrive at a technology for producing 2,5-furandicarboxylic acid and PEF in a commercially viable way.

2,5-Furandicarboxylic acid can be obtained by oxidation of molecules having furan moieties, e.g. 5-hydroxymethylfurfural (HMF) as well as the corresponding esters and ethers, e.g. 5-alkoxymethylfurfural and more specifically 5-methoxymethylfurfural (MMF), and similar starting materials, that are typically obtained from plant-based sugars, e.g. by sugar dehydration. A broad variety of oxidation processes is known from the prior art such as enzymatic and metal catalysed processes, either heterogeneous or homogeneous. WO 2014/014981 and WO 2011/043661 describe processes using catalyst systems comprising cobalt, manganese and bromine to oxidize compounds having a furan moiety to 2,5-furandicarboxylic acid using oxygen or air as an oxidizing agent.

The purity of crude 2,5-furandicarboxylic acid product is oftentimes not sufficient for use in the manufacture of polymers having desirable properties. The product obtained by oxidation can be coloured. Colour is disadvantageous in that it indicates the presence of impurities. In order to objectively determine colour, absorbance of a solution is to be measured at a particular wavelength such as 400 nm. Impurities which lead to colour are often difficult to specifically identify making their removal difficult.

Processes have been developed for further purifying crude oxidation products. Exemplary purification processes are disclosed in WO 2014/014981 and WO 2016/195499.

It can be advantageous to apply a post-oxidation step. WO2021/123206 describes that a subsequent oxidation reaction is suitable for converting the product of incomplete oxidation.

Unfortunately, it was found that the post-oxidation reaction can be erratic especially in continuous processes where oscillatory behavior was observed. During such oscillatory behavior oxygen consumption and carbon monoxide and/or carbon dioxide production changed periodically. It was found that oscillatory behavior of the post-oxidation reaction frequently ended in inadequate or no post-oxidation. 2,5-Furandicarboxylic acid obtained after inadequate or no post-oxidation was found to have an unfavorable color.

The article “Optimization of Co/Mn/Br-catalyzed oxidation of 5-hydroxymethylfurfural to enhance 2,5-furandicarboxylic acid yield and minimize substrate burning” by Xiaobin Zuo et al, ACS Sustainable Chemistry Engineering 2016, pages 3659-3668 studied furandicarboxylic acid formation by semicontinuous oxidation of 5-hydroxymethylfurfural. The conversion is described to be accompanied by competitive side reactions most notably esterification and overoxidation to carbon monoxide and carbon dioxide. Furandicarboxylic acid yield was improved by optimization of operating variables such as catalyst composition, water concentration in the acetic acid solvent and pressure.

U.S. Pat. No. 4,906,772 describes co-production of acetic acid from ethyl alcohol in a reaction to prepare terephthalic acid from p-xylene.

EP-A-2752446 relates to recycling of polyethylene terephthalate by ethanolysis to form ethyl esters and ethylene glycol and oxidizing the resulting ethyl ester to form carboxylic acid and acetic acid.

WO 2021/231556 describes a process for making 2,5-furandicarboxylic acid and terephthalic acid by co-feeding (i) 2,5-furandicarboxylic acid forming furanics such as 5-hydroxymethyl furfural and (ii) p-xylene. Post-oxidation was applied in the Examples on “Co-oxidation of p-xylene and FDCA-Forming Furanics with Co, Mn, Br catalysts”. The main oxidation reaction was allowed to proceed for a target run time before ceasing feed and allowing the reaction to proceed without additional substrate for a target post-oxidation time.

WO2021/123189 applies post-oxidation to decrease the amount of mono alkyl ester of 2,5-furandicarboxylic acid in mother liquor which otherwise tends to build up under certain conditions.

### DISCLOSURE OF THE INVENTION

The present invention concerns producing 2,5-furandicarboxylic acid by oxidation of a furan derivative followed by post oxidation.

The objective was to ensure that the post oxidation process remains active thereby allowing to obtain crude 2,5-furandicarboxylic acid of higher quality. A higher quality 2,5-furandicarboxylic acid contains less impurities or impurities which are less harmful in further use of 2,5-furandicarboxylic acid.

A further objective was to improve the colour of the 2,5-furandicarboxylic acid obtained, i.e. reduce the absorbance at 400 nm of the crude carboxylic acid obtained.

Surprisingly, it now has been found that post oxidation remains active by adding an oxidizable compound to its feed or directly to the reactor. Without wishing to be bound to any theory, it is thought that the oxidizable compound ensures that the catalyst system remains in a reduced state. Alternatively, it could be that the additional oxidizable compound ensures the presence of free radicals. Whatever is the mechanism, additional and thorough oxidation can reduce the amount of color forming bodies and/or compounds of the crude 2,5-furandicarboxylic acid which furthermore can cause retention of catalytic metals in the 2,5-furandicarboxylic acid.

The invention relates to a process for producing 2,5-furandicarboxylic acid which process comprises (i) contacting in an oxidation unit a furan derivative with oxidant and solvent in the presence of a catalyst system to obtain intermediate product comprising 2,5-furandicarboxylic acid, (ii) contacting in a post-oxidation unit the intermediate product with oxidant to which post-oxidation unit oxidizable compound is added to obtain crude product, and (iii) separating the crude product into 2,5-furandicarboxylic acid and mother liquor containing solvent wherein the oxidizable compound is selected from the group consisting of tetrahydrofuran containing compounds comprising of from 4 to 10 carbon atoms and alcohols comprising of from 2 to 8 carbon atoms and mixtures thereof.

### FIGURE

FIG. 1 shows a general concept of a process line-up of the present invention.

### MODES FOR CARRYING OUT THE INVENTION

The process is aimed at producing 2,5-furandicarboxylic acid. The product obtained can contain further compounds besides 2,5-furandicarboxylic acid such as side-products of the oxidation of the furan derivative. Well known side-products of the oxidation of HMF and/or MMF are for example 5-formyl-2-furancarboxylic acid (FFCA), monomethyl ester of 2,5-furandicarboxylic acid and methyl ester of FFCA. The monomethyl ester of 2,5-furandicarboxylic acid can be used in the preparation of polyesters by transesterification and therefore is considered a desirable end-product as well.

Step (i) comprises oxidation of a furan derivative with oxidant and solvent in the presence of a catalyst system.

The furan derivative can be any furan derivative known to be suitable. More specifically, the furan derivative is a furfural derivative and more specifically a furfural derivative selected from the group consisting of 5-methylfurfural (MF) and derivatives having the chemical formula (1)

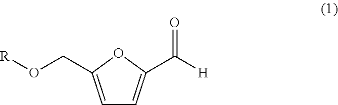

wherein R represents hydrogen, an alkyl group or an acyl group. The feed preferably comprises at least 80% by weight of MF, HMF and/or MMF, more preferably at least 90% by weight of MF, HMF and/or MMF. The feed preferably consists of HMF and/or MMF.

It is generally undesirable to have further compounds present which are to be oxidized such as phenyl containing compounds which are to be converted to terephthalic acid or a derivative thereof. Therefore, the reaction mixture of step (i) preferably is substantially free from phenyl containing compounds. More preferably, the reaction mixture of step (i) is free from phenyl containing compounds.

Besides the furan derivative, the mixture present in step (i) comprises oxidant, solvent and a catalyst system.

Preferably, the solvent is an organic acid, water or a mixture thereof. Preferably, the solvent comprises water and/or acetic acid. The solvent can be a recycle stream or can be freshly added. A stream which is especially suitable is mother liquor obtained from separation and/or purification of 2,5-furandicarboxylic acid slurry. More preferably, the solvent is selected from the group consisting of water, acetic acid and mixtures thereof. After the reaction has started, the amount of water present in the reaction mixture of step (i) tends to increase due to the further water produced by oxidation of the furan derivative.

The catalyst system comprises cobalt, manganese and bromine either as the element or as a derivative thereof. The catalyst system additionally tends to contain a limited amount of water. The catalyst system preferably has a weight ratio of cobalt to manganese in the catalyst system of 5 or higher, preferably 10 or higher, preferably 15 or higher, and/or a weight ratio of bromine to the combined weight of cobalt and manganese in the catalyst system of 1 or higher, preferably 1.5 or higher, most preferably 2 or higher, wherein the value is preferably less than 4.0, more preferably less than 3.5, more preferably less than 3, more preferably less than 2. If the catalyst system comprises other metals besides cobalt and manganese in an amount of 5% by weight or more, it is preferred that the above ratios are achieved for the weight ratio of bromine to the combined weight of all metals in the catalyst system. The metals preferably are added as salts which are soluble in the reaction mixture. Typically, the amount of cobalt is selected in the range of 500 to 6000 ppm by weight, based on the weight of the reaction mixture in the oxidation unit including furan derivative, solvent and catalyst system. The amount of manganese typically is in the range from 20 to 6000 ppm by weight, based on the weight of the reaction mixture in the oxidation unit including furan derivative, solvent and catalyst system Typically, the bromine concentration would be from 30 to 8000, preferably 50 to 4500 ppm by weight of bromine, based on weight of the reaction mixture in the oxidation unit including furan derivative, solvent and catalyst system. Alternatively, the bromine content is from 3000 to 8000 ppm by weight.

The oxidant for use in each step (i) and step (ii) can be any gas known to be suitable by the person skilled in the art. Preferably, the oxidant comprises molecular oxygen. Most preferably, the source of the oxidant is air.

It will be clear that the amount of oxidant required in step (i) depends on the furan derivative to be oxidized. The amount of oxidant in step (i) generally is 0.5 to 10 mol of oxygen per mole of furan derivative, preferably of from 1 to 8, more preferably of from 1 to 5 mole of oxygen per mole of furan derivative.

The current process can be carried out in batch or semi-batch, or continuously. The process is especially advantageous for continuous operation which will be allowed to continue uninterrupted due to sufficient post oxidation.

A process is considered to be carried out continuously if the furan derivative is added to the oxidation reactor substantially continuously. Preferably, the intermediate product additionally is added continuously to the post oxidation unit. More preferably, additionally the intermediate product is removed from the oxidation unit continuously and the crude product is removed from the post oxidation unit continuously. Preferably, the oxidizable compound is added intermittently or continuously to the post-oxidation unit if the intermediate product is added continuously. Most preferably, the oxidizable compound is added continuously. An operation is considered to be continuous if the flow continues for the majority of the time, preferably during at least 70% of the time. Operation is considered to be continuous even if the flow of fluid is frequently stopped for a short time fore example due to a valve regularly closing and opening again.

Step (i) preferably is carried out at a temperature in the range of 150 to 210° C., preferably a temperature of 160 to 190° C., more preferably a temperature in the range of from 165 to 180° C. Preferably, the pressure in step (i) is in the range of 6 to 20 barg. These parameters were found to be preferred for obtaining intermediate product of good purity in good yields.

The intermediate product tends to comprise mono alkyl ester of 2,5-furandicarboxylic acid besides 2,5-furandicarboxylic acid. Alkyl ester of 2,5-furandicarboxylic acid also is considered a desirable product as mentioned above.

The above temperature range allows the oxidation reactor to run at elevated pressure while still allowing the large amount of heat generated by the exothermic oxidation reaction to be removed by vaporization. This is known to one skilled in the art as “adiabatic” operation wherein heat of reaction is not being removed by sources such as coolers, loss through the walls, and the like but in majority by evaporation of the solvent and recycling of cold condensate. In general, the higher temperature requires a higher pressure for “adiabatic” operation. A higher pressure, in turn, allows for a higher oxygen partial pressure in the reactor and reduces the risk of oxygen starvation. The latter means that the reaction is limited by the oxygen available.

The unit for carrying out the oxidation can be any typical oxidation reactor that is known in the art. The oxidation unit of step (i) preferably is a continuously stirred tank reactor.

Part or all of the intermediate product obtained in step (i) is subjected to post-oxidation in step (ii). At least part of the gaseous product of step (i) can be removed as off-gas. Such gaseous stream can separated and subsequently be cooled. It is possible to separate off compounds such as acetic acid from the intermediate product to be sent back to step (i) or to other parts of the process.

In a preferred embodiment, gaseous intermediate product and/or gaseous crude product is separated off, cooled and recycled to step (i). The oxygen volume percent in the off-gas is generally limited for safety reasons to be below the lower explosive limit, e.g. at a level below 10 vol %, or more preferably below about 6 vol % to allow a margin of safety. Oxygen containing gas can be recycled to step (i) or used in step (ii) or recycled or used in other parts of the process. As it is low in oxygen, it can be used as a conveying gas or for running the drier and/or filter.

It is preferred to submit all of the reaction slurry produced in step (i) to step (ii), more specifically all of the catalyst system present in step (i). The slurry consists of the solids and liquids of the intermediate product. All of the reaction slurry preferably is submitted to step (ii) by not separating either liquids or solids from the intermediate product. Submitting all of the reaction slurry of step (i) to step (ii) ensures that the catalyst system of step (i) also is of use in step (ii).

The post-oxidation of step (ii) preferably is applied at a temperature in the range of 150 to 210° C., more specifically of 160 to 210° C. Preferably, the pressure in step (ii) is in the range of 6 to 20 barg.

Surprisingly, it now has been found that post oxidation proceeds more efficiently if an oxidizable compound is added to the feed or directly to the reactor during post oxidation. The oxidizable compound is in addition to any intermediate product which is obtained by oxidation of a furan derivative and in addition to any other compound already present during the first oxidation.

The oxidizable compound is selected from the group consisting of tetrahydrofuran containing compounds comprising of from 4 to 10 carbon atoms and alcohols comprising of from 2 to 8 carbon atoms and mixtures thereof. The tetrahydrofuran containing compounds most preferably comprise 4 carbon atoms. Preferably, the oxidizable compound is selected from the group consisting of ethanol and tetrahydrofuran, more preferably is ethanol. Addition of ethanol has the advantage that its oxidation product acetic acid can act as solvent for the process. Ethanol can be added as such or in the form of an ethyl ester which forms ethanol at the reaction conditions of the post-oxidation unit. Most preferably, ethanol is added as such.

The amount of oxidizable compound present in step (ii) preferably is least 0.01% by weight based on total amount of liquid intermediate product, more preferably at least 0.05% by weight, more preferably at least 0.1% by weight. The amount of oxidizable compound preferably is at most 7% by weight based on total amount of liquid intermediate product present in the post-oxidation unit, more preferably at most 5% by weight, more preferably at most 3% by weight, more preferably at most 2% by weight. The amount of intermediate product is the total liquid and/or solid reaction mixture so including both the oxidized furan derivative and the solvent.

Preferably, the oxidant, preferably oxygen, added to step (ii) is at least 0.001 mole of oxidant per mole of 2,5-furandicarboyxlic acid and esters therefore. Preferably this ratio is at least 0.05. Preferably, this ratio is at most 5, more preferably at most 3, more preferably at most 2.

Preferably, at least part of the gaseous crude product is removed. Such gaseous crude product can be separated and be cooled before separating off compounds such as acetic acid which can be sent back to step (i), to step (ii) or to another part of the process.

The crude product obtained in step (ii) tends to be a slurry which comprises solid 2,5-furandicarboxylic acid and a liquid comprising solvent and water and dissolved catalyst system and possibly other organic impurities.

In step (iii) 2,5-furandicarboxylic acid can be separated from the crude product in a solid-liquid separation zone to obtain a solid cake and mother liquor. The separation of step (iii) can be carried out in any way known to the person skilled in the art. Preferred is a process wherein the solid-liquid separation zone comprises a filter or centrifuge, preferably a filter, more preferably a rotary pressure filter. The separation preferably is carried out by filtration. Not all of the 2,5-furandicarboxylic acid generally will be removed from the crude carboxylic acid composition while generally not all of the solid cake which is separated will be 2,5-furandicarboxylic acid.

The 2,5-furandicarboxylic acid obtained in step (iii) can be treated or washed with solvent selected from the group consisting of water and mono- and/or dicarboxylic acids. The acid preferably is a mono-carboxylic acid containing of from 1 to 3 carbon atoms.

In a preferred embodiment, the process further comprises (iv) contacting the 2,5-furandicarboxylic acid with polar solvent to obtain a solution; (v) contacting the solution with hydrogen in the presence of a hydrogenation catalyst at hydrogenation conditions yielding a hydrogenated solution; and (vi) separating purified 2,5-furandicarboxylic acid from the hydrogenated solution. Suitable process conditions are for example described in WO2016/195499. Preferred process conditions comprise contacting with hydrogen at a temperature in the range of 150 to 200° C. and a contact time with the hydrogenation catalyst in the range of 5 seconds to 15 min. Preferably, the polar solvent is selected from the group consisting of water, acetic acid and mixtures thereof.

It will be clear to the person skilled in the art that preferably all solution obtained in step (v) is subjected to step (vi) although it is possible to use part of the solution only.

The 2,5-furandicarboxylic acid obtained in step (iii) or step (vi) preferably comprises at least 50% by weight with respect to the weight of the dry cake of 2,5-furandicarboxylic acid, more preferably at least 70% by weight, more preferably at least 80% by weight, more preferably at least 90% by weight, most preferably at least 95% by weight. The wet cake can contain residual mother liquor. The dry cake can comprise derivatives of 2,5-furandicarboxylic acid such as ester of 2,5-furandicarboxylic acid, 2-carboxy-5-(formyl)furan (FFCA) and bis-carbonyl-furoic acid (BCFCA).

The crude 2,5-furandicarboxylic acid obtained in step (iii) preferably is purified by a process as specifically described in WO2021/123206 or WO2021/123203.

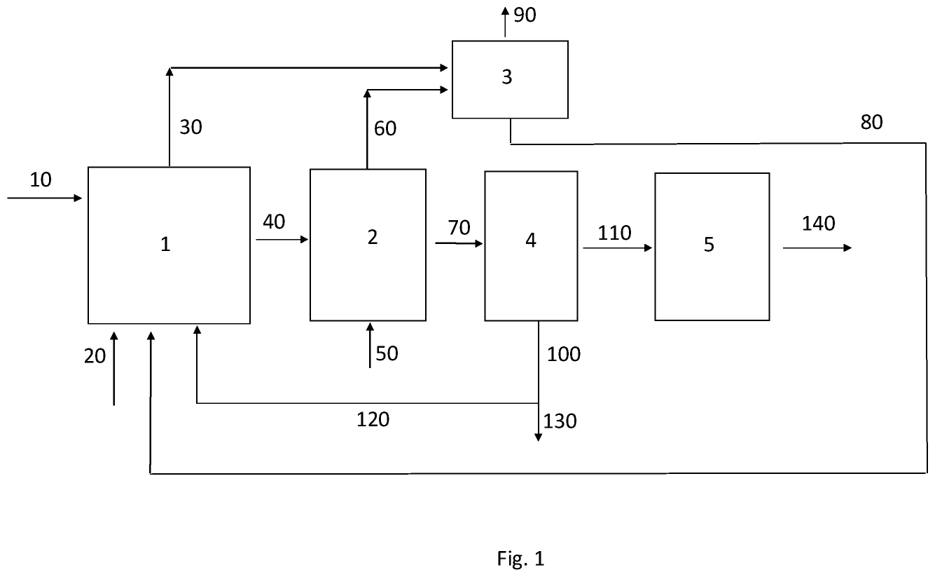

FIG. 1 shows a general concept of a process line-up of the present invention.

In the process of FIG. 1, a feed comprising hydroxymethyl furfural and/or methoxymethyl furfural in combination with solvent and catalyst system is fed via line 10 to oxidation unit 1. Separately, oxidant is added to unit 1 via line 20. Off-gas can be removed over the top via line 30 while liquid and solid intermediate products optionally with part of the gaseous intermediate product can be sent via line 40 to post oxidation unit 2. Air is added to post oxidation unit 2 via line 50. Oxidizable compound either pure or mixed with solvent can be added to line 40 (by an addition point not shown) to enter the post oxidation unit 2 mixed with feed or is added directly via line 60 to the post oxidation unit 2. Gaseous crude product can be removed via line 70. Off-gas of the oxidation unit can be sent via line 30 and/or off-gas of the post-oxidation unit can be sent via line 70 to unit 3 where these gaseous effluents can be cooled and optionally further treated to obtain a liquid solvent stream which can be recycled to oxidation unit 1 via line 80 or to other parts of the process (not shown). Off-gas of unit 3 can be removed via line 90. Liquid and solid crude products can be sent via line 100 to a separator unit 4 to obtain 2,5-furandicarboxylic acid cake and mother liquor. It may be preferred to cool liquid and solid crude products between the post oxidation step and the separator step. Mother liquor can be removed via line 120. The 2,5-furandicarboxyl acid cake can be sent via line 110 to unit 5 for further purification. Purified 2,5-furandicarboxyl acid can be removed via line 130.

Hereinafter, the invention is described in more detail in a non-limiting example.

### Example

The current experiment was carried out as part of a continuous operation.

Liquid feed for the oxidation reactor was prepared by sugar dehydration. The liquid feed comprised as furan derivatives 18% wt 5-methoxymethylfurfural and 1.4% wt 5-hydroxymethyl furfural and as solvent a mixture of a major amount of acetic acid and a minor amount of water. The liquid feed also comprised minor amounts of by-products of the sugar dehydration including levulinic acid and methyl levulinate. Further, the liquid feed comprised an oxidation catalyst system composed of cobalt acetate hydrate, manganese acetate hydrate and hydrobromic acid. The oxidant fed into the reactor was air. The molar ratio of oxygen per hour to the combined molar amount of 5-methoxymethylfurfural and 5-hydroxymethyl furfural per hour was about 4.

Liquid feed and oxidant were fed continuously to a continuously stirred tank reactor in which the liquid feed was contacted with oxygen at 175° C. and 11 barg at a residence time of about 1.2 hour.

The liquid slurry produced in the oxidation reactor was fed continuously into a post-oxidation reactor where the slurry was contacted at 165° C., a pressure of 10 barg and air at a molar ratio of 0.4 of mole per hour of oxygen to the combined molar amount per hour of furandicarboxylic acid and monomethyl furandicarboxylate. Residence time was 1.2 hour. Additionally, ethanol was fed to the post oxidation reactor in an amount of about 0.2 mol per hour of ethanol to the combined molar amount of furandicarboxylic acid and monomethyl furandicarboxylate per hour.

Vapors from the oxidation and post-oxidation reactor were removed overhead and condensed. The vapor fractions were partly recycled to the oxidation reactor.

Activity of the post oxidation reactor was monitored by oxygen consumption and production of carbon dioxide and carbon monoxide.

After 231 hours of continuous operation, the ethanol flow to the post-oxidation reactor was stopped. Subsequently, a pulse of ethanol was added every two hours to check for activity. The reaction was considered to be still active if the pulse of ethanol was followed by a spike in oxygen consumption. If there was no response, it was assumed that there no longer was post oxidation activity. The amount of oxygen consumed and the amount of carbon dioxide and carbon monoxide produced started to fluctuate after the ethanol started to be added as pulse.

After 250 hours of operation (so 19 hours after stopping the addition of ethanol), the ethanol pulse did no longer give rise to an increase in oxygen consumption. It was concluded that activity in the post oxidation reactor had stopped.

It was possible to later restart the post oxidation reactor (in that oxygen consumption and carbon dioxide and carbon monoxide production started again) by a pulse of methoxymethyl furfural.

The crude product of the post oxidation reactor was fed to a filter to obtain a 2,5-furandicarboxylic acid cake. It was found that the colour of this filter cake increased considerably when the ethanol pulse did no longer give rise to an increase in oxygen consumption i.e. the post oxidation was not active. The colour was determined by absorbance of a solution of the filter cake at a wavelength of 400 nm.

1. A process for producing 2,5-furandicarboxylic acid which process comprises
(i) contacting in an oxidation unit a furan derivative with oxidant and solvent in the presence of a catalyst system to obtain intermediate product comprising 2,5-furandicarboxylic acid,
(ii) contacting in a post-oxidation unit the intermediate product with oxidant to which post-oxidation unit oxidizable compound is added to obtain crude product, and
(iii) separating the crude product into 2,5-furandicarboxylic acid and mother liquor containing solvent,
wherein the oxidizable compound is selected from the group consisting of tetrahydrofuran containing compounds comprising of from 4 to 10 carbon atoms and alcohols comprising of from 2 to 8 carbon atoms and mixtures thereof.

2. The process according to claim 1 wherein the oxidizable compound is selected from the group consisting of ethanol and tetrahydrofuran, more preferably is ethanol.

3. The process according to claim 1 wherein the amount of oxidizable compound is of from 0.01% to at most 7% by weight based on total amount of liquid intermediate product.

4. The process according to claim 1 wherein the furan derivative has the chemical formula (1) 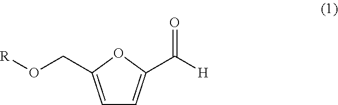
wherein R represents hydrogen, an alkyl group or an acyl group.

5. The process according to claim 1 wherein the process further comprises:
(iv) contacting the 2,5-furandicarboxylic acid with polar solvent to obtain a solution;
(v) contacting the solution with hydrogen in the presence of a hydrogenation catalyst at hydrogenation conditions yielding a hydrogenated solution; and
(vi) separating purified 2,5-furandicarboxylic acid from the hydrogenated solution.

6. The process according to claim 1 wherein the process is continuous and furan derivative is added continuously to the oxidation unit.
